(19) United States
(12) Patent Application Publication
(10) Pub. No.: US 2024/0000558 A1
(43) Pub. Date: Jan. 4, 2024

(54) Controllable operation-free take-out ureteral stent with degradable coating

(71) Applicant: Jiahao JIN, Jiaxing (CN)

(72) Inventors: Jiahao JIN, Jiaxing (CN); Weiwen YU, Jiaxing (CN); Dahong ZHANG, Jiaxing (CN); Gonghui LI, Jiaxing (CN); Fuqing TAN, Jiaxing (CN); Jun LU, Jiaxing (CN); Guohai XIE, Jiaxing (CN); Xiang HE, Jiaxing (CN)

(73) Assignee: Jiahao JIN, Jiaxing (CN)

(21) Appl. No.: 18/369,867

(22) Filed: Sep. 19, 2023

Related U.S. Application Data
(63) Continuation-in-part of application No. PCT/CN2021/094073, filed on May 17, 2021.

(30) Foreign Application Priority Data
Mar. 19, 2021 (CN) 202110296089.0
Apr. 20, 2021 (CN) 202110421734.7

(51) Int. Cl. A61F 2/04; A61M 27/00; A61L 27/34

(52) U.S. Cl. CPC A61F 2/04; A61M 27/008; A61L 27/34; A61F 2002/048; A61F 2230/0041; A61M 2205/0238; A61M 2210/1078; A61L 2430/22

(57) ABSTRACT

A controllable operation-free take-out ureteral stent with a degradable coating includes a ureteral stent, a traction wire, a discharged body, and a degradable fixture, where the discharged body is fixed on one end of the traction wire, and the degradable fixture fixes the discharged body on the other end of the traction wire and is arranged close to one end of the ureteral stent within a limited period of time; the ureteral stent comprises a nondegradable base layer and a degradable coating which are sequentially arranged from inside to outside; the problem of bacterial adhesion on the surface of the ureteral stent caused by long indwelling time or patients' special allergies can be solved by automatic degradation and peeling off of the degradable coating on the surface of the ureteral stent within a certain period of time.

### CROSS REFERENCE TO THE RELATED APPLICATIONS

This application is a continuation-in-part application of International Application No. PCT/CN2021/094073, filed on May 17, 2021, which is based upon and claims priority to Chinese Patent Applications No. 202110296089.0, filed on Mar. 19, 2021, and No. 202110421734.7, filed on Apr. 20, 2021, the entire contents of which are incorporated herein by reference.

### TECHNICAL FIELD

The present application relates to the technical field of ureteral stents, in particular to the technical field of controllable non-surgical take-out ureteral stents with a degradable coating.

### BACKGROUND

The ureter, which connects the renal pelvis on the upper side and the bladder on the lower side, is a pair of slender tubes with an average diameter of 0.5-0.7 cm and a total length of 25-35 cm. It is located behind the peritoneum and descends vertically into the pelvis along the front of the medial side of psoas major. A ureteral stent has the advantages of relieving ureteral obstruction, facilitating the discharge of calculus and promoting the postoperative recovery of ureter, and thus is widely used in hospitals.

The surface of the existing ureteral stent is easy to adhere to bacteria, and then form biofilm and even calculus shell. For this reason, some people in the industry choose to directly add a drug coating on the outer wall of the ureteral stent, so as to achieve antibacterial effect by using drugs, for example a drug coating composition for ureteral stent, preparation method and use thereof in CN109288832B. However, once the drug is completely released, the ureteral stent has no inhibitory ability. In addition, at present, the commonly used material of a ureteral stent is polyurethane or silica gel. Among them, the biocompatibility of polyurethane material is poor, which leads to the formation of urinary salts on the outer wall of the ureteral stent, which causes the ureteral stent to be blocked or even difficult to take out. Although the biocompatibility of a silicone material is better than a polyurethane material, it has high bacterial adhesion rate and high friction coefficient, which is not easy for doctors to operate. Therefore, CN215228881U discloses that a ureteral stent with a silica gel hydrophilic coating adopts a three-layer structure of hydrophilic coating, silica gel coating and polyurethane from the outside to the inside, so as to give consideration to the strength, smoothness and biocompatibility of the ureteral stent. However, it still cannot fundamentally prevent the accumulation of urinary salts on the surface of ureteral stent, nor can it prevent the formation of biofilm or calculus shell.

In addition, when the existing ureteral stent is left in the body, it is easy to cause urine reflux due to the increase of bladder pressure during urination, which in turn causes waist swelling and discomfort. Usually, there are problems such as long length, hard texture, poor histocompatibility and large size of curled J-shaped head in the bladder, and especially when the J-shaped head exceeds the bladder midline, it tends to cause discomfort to patients.

With the extensive development of minimally invasive surgery such as ureteroscopy, rigid ureteroscopy, percutaneous nephroscope, laparoscopy and robotic laparoscopy, the postoperative recovery speed of patients is getting faster and faster, so the requirements of patients are getting higher and higher. However, the existing double J-tube must be taken out by cystoscopy after placement, which greatly increases the pain of postoperative patients, especially for male patients, and also increases the medical expenses of patients. In order to solve the above technical problems, absorbable stents and in vitro suture method have been studied in the industry, but the effect is not ideal. China patent ZL201921377413.6 “A New Ureteral Stent Conveniently Removed Without Operation” solved the technical problem that the ureteral stent in the prior art had to be removed by cystoscope surgery and could not be discharged by itself by a wire-ball combination method. However, it is only suitable for patients who have been placed with double J tubes for a short time because the time of discharging the ball during urination is uncontrollable and the discharge time is short.

### SUMMARY

The object of this application is to solve the problems in the prior art, and put forward a controllable operation-free take-out ureteral stent with a degradable coating, the problem of bacterial adhesion on the surface of the ureteral stent caused by long indwelling time or patients' special allergies can be solved by automatic degradation and peeling off of the degradable coating on the surface of the ureteral stent within a certain period of time, thus avoiding the formation of biofilm or even calculus scale on the surface of the ureteral stent; further, different degradable fixtures can also be used to realize the relatively accurate control of the time for releasing the fixation of the discharged body, so as to meet the time controllability requirement of pulling out the ureteral stent without surgery in cooperation with the traction wire. At the same time, the indwelling part in the bladder of the ureteral stent is reduced as much as possible, and an anti-reflux water-retaining soft sleeve is added to prevent reflux, and the hardness of the ureteral stent gradually softens from one end close to the human renal pelvis to one end close to the human bladder. The diameter also gradually narrows from one end close to the human renal pelvis to one end close to the human bladder, effectively avoiding the discomfort caused by the increase of bladder pressure and urine reflux during urination, such as waist swelling pain, back pain caused by indwelling ureteral stent and bladder irritation symptoms.

The discomfort such as waist swelling and pain and bladder irritation caused by the increase of bladder pressure and urine reflux during urination are effectively avoided.

In order to achieve the above object, the present application puts forward a controllable operation-free take-out ureteral stent with a degradable coating, including a ureteral stent, a traction wire, a discharged body and a degradable fixture, wherein the discharged body is fixed on one end of the traction wire, and the degradable fixture fixes the discharged body on the other end of the traction wire and is arranged close to one end of the ureteral stent within a limited period of time; the ureteral stent includes a nondegradable base layer and a degradable coating which are sequentially arranged from inside to outside.

Preferably, the nondegradable base layer is made of polyurethane or silica gel, and the degradable coating layer is made of a degradable material selected from polylactic acid, polyglycolic acid, polylactic-glycolic acid copolymer, chitosan, alginate-based material, polyhydroxyalkanoate and polydioxanone.

Further, the polyurethane can be modified polyurethane, preferably aliphatic polyurethane, such as (Tecofex), which can be developed under X-ray.

Further, the degradable coating can be a composite material of poly-L-lactic acid, polyglycolic acid and caprolactone.

Further, one of hydrophilic substance, antibacterial and anti-inflammatory substance and anticoagulant substance is mixed in the degradable coating, wherein the hydrophilic substance is one of polyvinylpyrrolidone, sodium hyaluronate and hydrogel; the antibacterial and anti-inflammatory substance is one of triclosan, silver sulfadiazine salt, sirolimus and rifampicin; and the anticoagulant substance is heparin sodium; specifically, when only hydrophilic substances are mixed in the degradable coating, a degradable hydrophilic mixed coating can be formed together; when only antibacterial substances are mixed in the degradable coating, a degradable antibacterial coating can be formed together; when only anticoagulant substances are mixed in the degradable coating, a degradable anticoagulant coating can be formed together.

Preferably, a hydrophilic coating is also arranged between the nondegradable base layer and the degradable coating.

Further, the hydrophilic coating is composed of one of polyacrylamide, polyvinylpyrrolidone, polyoxyethylene, hydrogel and sodium hyaluronate.

Preferably, the ureteral stent includes a stent body and a forward J-shaped tube, wherein the stent body is tubular and one end thereof far from the discharged body is be connected with the forward J-shaped tube; a drainage channel runs through the stent body and the forward J-shaped tube, the drainage channel is located in the nondegradable base layer and runs through the front and back, and a plurality of drainage holes communicating with the outside and the drainage channel are respectively arranged on the stent body and the forward J-shaped tube.

Preferably, one end of the stent body close to the discharged body is further provided with a bladder retaining structure, which is a flared tube, an elliptical tube or an inverted J-shaped tube, and a ring size of the inverted J-shaped tube is smaller than that of the forward J-shaped tube. The specific shape of the bladder retaining structure can be selected according to the specific conditions of hydronephrosis, ureter and bladder during operation.

Preferably, the other end of the bladder retaining structure is also connected with one end of an anti-reflux water-retaining soft sleeve, and a length of the anti-reflux water-retaining soft sleeve is not less than twice a width of the bladder retaining structure.

Further, the anti-reflux water-retaining soft sleeve is preferably made of an extremely soft material, which can be in a free state during urine storage, so that urine in the renal pelvis can smoothly enter the bladder, cover the bladder retaining structure under the action of bladder pressure during urination, prevent urine in the bladder from flowing back to the renal pelvis, and return to a free state after urination is completed and bladder pressure is reduced.

Still further, a plurality of marking scales for length measurement are arranged outside the stent body, and the hardness of the forward J-shaped tube, the stent body and the bladder retaining structure gradually becomes softer, and the diameters of the forward J-shaped tube and the stent body gradually becomes smaller. This can further increase the natural space of ureter after indwelling ureteral stent, which is beneficial to the natural drainage of ureter and reduces discomfort.

Still further, the marking scale includes a plurality of positioning rings with different intervals and different styles which are sequentially arranged along the length direction of the stent body. Specifically, at least one of the ring number, ring width or ring color of each positioning ring is different, and a first positioning ring is arranged at the joint of the stent body and the forward J-shaped tube, a second positioning ring is arranged at a distance of 10-20 cm from the first positioning ring, and the next positioning ring is arranged every 2-8 cm thereafter. In addition, a ruler scale can be set between two adjacent positioning rings at an interval of 0.5-2 cm.

Preferably, the degradable fixture is one or more of degradable wire, degradable perforated module and medical glue; the degradable wire and the degradable perforated module are both binary copolymers formed by polymerization of glycolide and lactide, terpolymers or alginate polymers formed by polymerization of glycolide, lactide and caprolactone, and the medical glue is a-cyanoacrylate. Specifically, the degradable wire is preferably a copolymer of 90%-98% glycolide and 10%-2% L-lactide, in which the higher the proportion of glycolide, the smaller the molecular weight and the faster the degradation speed, for example a copolymer (Polyglactin910) of 90% glycolide and 10% L-lactide, that is, Vicryl Rapide, Polyglactin 910 of Johnson & Johnson Company, the absorption time of which in vivo is 42 days. According to the discharge time requirement of the discharged body, other materials that meet the degradation time can also be used to make degradable fixtures. In addition, the degradation time of the degradable fixture is influenced by many factors, such as the degradation properties of the material of the degradable fixture (the proportion and molecular weight of different degradable materials, etc.), the size of degradable fixture, and the combined use modes of different types of degradable fixtures. Therefore, by adjusting the above factors, the fixture releasing time of the discharged body can be controlled relatively accurately, so as to realize the time controllability requirement of the operation-free pulling out of the ureteral stent.

Further, the length of the degradable wire is 1-5 cm and the thickness is 3-50 zeros, the degradable perforated module is spherical or tubular with holes, and the length and/or outer diameter of the degradable perforated module is 0.5-3 mm.

Still further, a length of the traction wire is longer than that of a human urethra, the traction wire is a non-absorbent and nondegradable wire, and some points on a line segment of the traction wire are simultaneously fixed by degradable wires and/or medical glue to be folded.

Still further, the surface of the traction wire is provided with an antibacterial coating.

Still further, the antibacterial coating consists of one of triclosan, silver sulfadiazine salt and rifampicin.

Still further, a surface of the discharged body is smooth and provided with a plurality of through holes, a density of the discharged body is higher than that of water; both the traction wire and the degradable wire can pass through the through holes to bind the discharged body, and a largest through hole has a hole size allowing a loach guide wire or a zebra guide wire to pass through. Wherein, each through hole can be communicated with each other or not, so that the traction wire and the degradable wire can pass through the same or different through holes.

Still further, the time-limited fixation of the discharged body and the time-limited folding of the traction wire are independently completed by the degradable wire. Specifically, when the degradable wire is used, it is in the form of a knot. On the one hand, the degradable wire binds the discharged body and fixes it on one end of the traction wire near the ureteral stent, and on the other hand binds the traction wire into a folded shape.

Still further, the time-limited fixation of the discharged body and the time-limited folding of the traction wire are independently completed by medical glue. Specifically, on the one hand, the medical glue sticks the discharged body to one end of the traction wire close to the ureteral stent, and on the other hand, sticks the traction wire into a folded shape.

Still further, the time-limited fixation of the discharged body and the time-limited folding of the traction wire are jointly completed by the degradable wire and the medical glue. Specifically, the degradable wire binds and fixes the discharged body on one end of the traction wire close to the ureteral stent, and the medical glue simultaneously sticks the traction wire into a folded shape.

Still further, the time-limited fixing of the discharged body and the time-limited folding of the traction wire are jointly completed by the degradable perforated module and the medical glue. Specifically, the traction wire can pass through the tube hole of the degradable perforated module to bind and fix the discharged body on one end of the traction wire close to the ureteral stent, and the medical glue simultaneously sticks the traction wire into a folded shape.

Still further, the discharged body is spherical with through holes and has no magnetism. Specifically, the discharged body is preferably made of 304 stainless steel or martensitic stainless steel or ferritic stainless steel which can be attracted by magnetic substances.

Furthermore, the discharged body is spherical with through holes and magnetic. Specifically, the discharged body is preferably made of neodymium magnets. In addition, an anti-corrosion coating can be added to the surface of the discharged body made of neodymium magnet. When the degradable fixture reaches the required degradation time, if it is not completely degraded, it can be attracted by the magnet outside the body, so that the discharged body can break free from the bondage of the degradable fixture by the attraction of the magnet outside the body and be discharged within a predetermined period of time.

The present application has the beneficial effects that:

1. By dividing the ureteral stent into two layers, on the one hand, the internal main body material is made of nondegradable polyurethane or silica gel, and on the other hand, a degradable coating is arranged on the external surface of the main body material, so that the bacterial adhesion on the surface of the ureteral stent caused by the prolonged indwelling time or the patient's special allergic constitution can be solved by gradually degrading, absorbing or falling off the degradable coating within a certain period of time after the ureteral stent is implanted, thereby avoiding the formation of biofilm or even calculus scale on the surface of the ureteral stent, and at the same time, the degradable coating is very thin, and the degradation and absorption products are extremely harmful to human body.

2. According to the application, the hydrophilic coating is added between the nondegradable base layer and the degradable coating, so that after the degradable coating is absorbed or falls off, the surface of the ureteral stent can still maintain good hydrophilicity, thereby effectively improving the comfort of ureteral stent implantation.

3. In the application, one of hydrophilic substance, antibacterial and anti-inflammatory substance and anticoagulant substance is mixed into the degradable coating at the same time, so that the hydrophilic substance can be used to increase the hydrophilicity of the degradable coating, thereby reducing the resistance when the ureteral stent is implanted, and the antibacterial and anti-inflammatory substance and anticoagulant substance can be used to further avoid the formation of calculus scale and bacterial biofilm on the surface of the ureteral stent.

4. Under the condition of the existing equipment, the application can very conveniently complete the pushing and placing of the ureteral stent, traction wire, discharged body and degradable fixture at one time through the guide wire and the push rod, and at the same time, by selecting one or more of degradable wire, degradable perforated module and medical glue as the degradable fixture, and by using the regulation of the degradation properties of the degradable fixture material and the size of the degradable fixture and other factors, the degradation time of the degradable fixture is more accurately controlled, and the fixation releasing time of the discharged body is relatively accurately controlled to meet different clinical requirements, so that when the discharged body is automatically discharged from the body under the action of its own gravity and urine impact force, the ureteral stent is directly pulled out of the body with the traction wire connected with it, and the secondary cystoscope operation is not needed. The overall structure is simple, the cost is low, the operation is convenient, the pain and economic burden brought to patients by the secondary cystoscope operation are effectively avoided. Moreover, the body of the degradable fixture is small, and the degradation products in the bladder are rarely produced, which is easy to be discharged with urine and has no great discomfort to the human body.

5. According to the application, the hardness of the ureteral stent is gradually softened from one end close to the human renal pelvis to one end close to the human bladder, and the diameter is gradually reduced; at the same time, a special bladder retaining structure is added at one end of the ureteral stent with a discharged body, even if the ureteral stent is a single J tube and the end close to the discharged body does not extend out of the ureter, the indwelling part in the bladder without the ureteral stent is reduced as much as possible, and at the same time, a water-retaining soft sleeve is added at the indwelling part in the bladder to prevent backflow, which effectively avoids discomfort such as waist swelling pain and bladder irritation caused by urine reflux due to increased bladder pressure during urination, so as to jointly reduce discomfort after ureteral stent placement.

6. According to the application, the middle point or multiple points of the traction wire can be folded and fixed by using the degradable fixture, so that the traction wire exposed in the bladder can be divided into six or more strands, which is convenient for being sent into the body; and because the traction wire is a non-absorbent wire, the problem of urine leakage caused by siphon phenomenon of the traditional silk wire can be avoided when being discharged into the posterior urethra, and the operation is simple, easy to master, no special instruments are needed, and the manufacture is also very simple, so that the traction wire can be industrialized and popularized on a large scale, thereby alleviating the pain of patients and reducing the medical expenses of patients.

The features and advantages of the present application will be described in detail through examples and attached drawings.

### BRIEF DESCRIPTION OF THE DRAWINGS

FIG. 1 is a front view of Embodiment 1 when the degradable fixture is not degraded;

FIG. 2 is a front view of Embodiment 1 when the degradable fixture has been degraded;

FIG. 3 is a schematic cross-sectional view of the stent body or the forward J-shaped tube in Embodiment 1 where there is no drainage hole;

FIG. 4 is a front sectional view of the discharged body of Embodiment 1;

FIG. 5 is a use state diagram of Embodiment 1 when the discharged body is not discharged from the human body;

FIG. 6 is a use state diagram of Embodiment 1 when the discharged body has been discharged from the human body;

FIG. 7 is a schematic cross-sectional view of the stent body or the forward J-shaped tube in Embodiment 2 where there is no drainage hole;

FIG. 8 is a front view of Embodiment 3 when the degradable fixture is not degraded;

FIG. 9 is a front view of Embodiment 4 when the degradable fixture is not degraded;

FIG. 10 is a front view of Embodiment 5 when the degradable fixture is not degraded;

FIG. 11 is a front view of Embodiment 6 when the degradable fixture is not degraded;

FIG. 12 is a front view of Embodiment 7 when the degradable fixture is not degraded;

FIG. 13 is a front view of Embodiment 8 when the degradable fixture is not degraded;

FIG. 14 is a front sectional view of the discharged body of Embodiment 9.

In the figures: 1—Ureteral stent, 11—Stent body, 12—Forward J—tube, 13—Nondegradable base, 14—Degradable coating, 15—Hydrophilic coating, 16—Drainage channel, 17—Drainage hole, 18—Bladder retaining structure, 2—Traction wire, 3—Discharged body and 31—Through hole.

### DETAILED DESCRIPTION OF THE EMBODIMENTS

### Embodiment 1

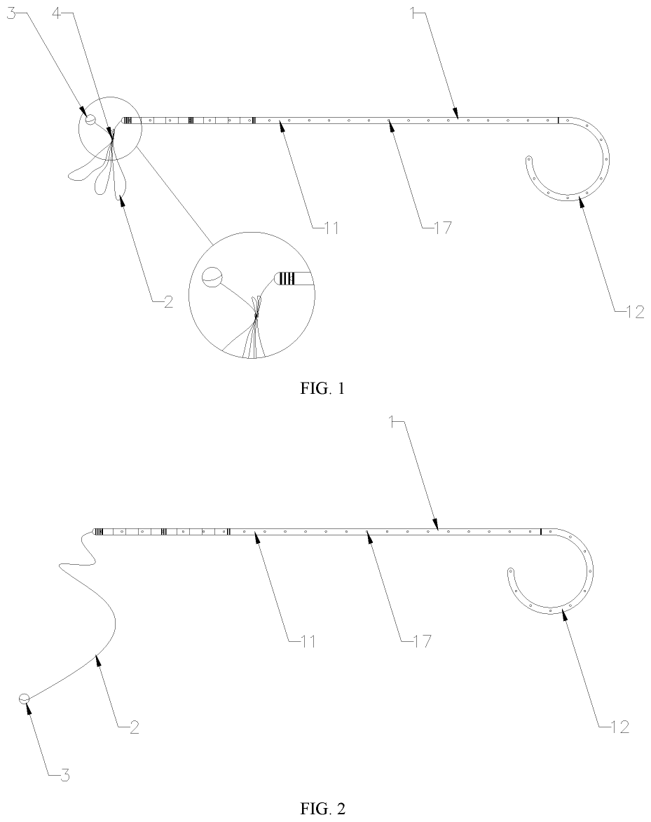

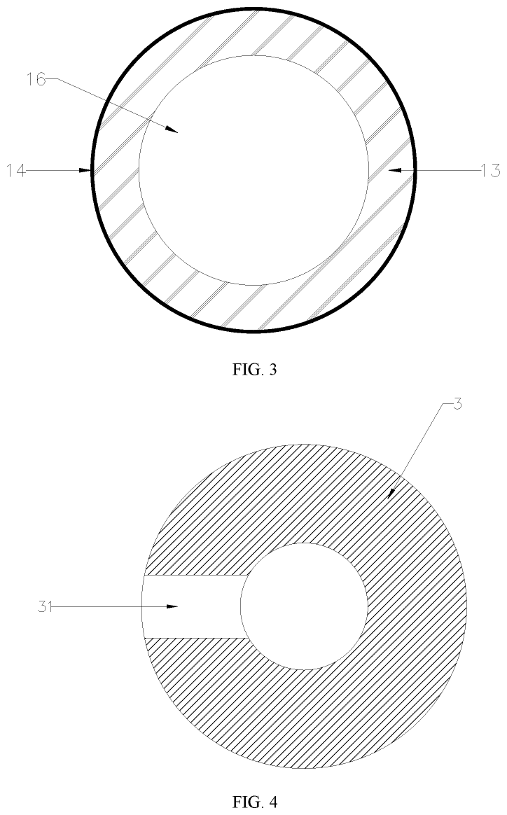

Referring to FIG. 1 to FIG. 6, the present application relates to a controllable operation-free take-out ureteral stent with a degradable coating, which includes a ureteral stent 1, a traction wire 2, a discharged body 3 and a degradable fixture 4, wherein the discharged body 3 is fixed on one end of the traction wire 2, and the degradable fixture 4 fixes the discharged body 3 on the other end of the traction wire 2 within a limited period of time and is arranged close to one end of the ureteral stent 1; the ureteral stent 1 includes a nondegradable base layer 13 and a degradable coating layer 14 sequentially arranged from inside to outside, that is, the ureteral stent 1 has a two-layer structure. Among them, the degradable fixture 4 can prevent the discharged body 3 from being discharged from the body prematurely under the impact of gravity and urine when urinating, and when the degradable fixture 4 is degraded, the discharged body 3 will be automatically discharged from the body under the impact of gravity and urine. At this time, medical staff or patients can directly pull the ureteral stent 1 out of the body by pulling the discharged body 3, thus avoiding secondary cystoscopy surgery. In addition, in this embodiment, because there is no bladder retaining structure 18, it is a single J-tube, so that the end of the stent body 11 close to the discharged body 3 is directly straight and located in the ureter, and the traction wire 2, the discharged body 3 and the degradable fixture 4 are all in the bladder, and the traction wire 2 is tied into six strands at two points. Since only the traction wire 2 passes through the openings of the bladder and ureter, the muscle anti-reflux effect at the sneaking opening of the bladder and ureter is not affected, so the foreign body sensation after the ureteral stent is implanted, and due to natural anti-reflux, it is very suitable for patients with sensitive constitution. Moreover, during implantation, the degradable coating 14 can gradually degrade and fall off with the passage of time, thus effectively preventing bacteria from forming biofilm on the surface of the ureteral stent 1, and even adhering to calculus scale. At the same time, the degradable coating 14 is very thin, and there are very few degradation products, which are easily discharged with urine and have no great discomfort to human body.

The nondegradable base layer 13 is made of polyurethane or silica gel, and the degradable coating layer 14 is made of a degradable material selected from polylactic acid, polyglycolic acid, polylactic acid-glycolic acid copolymer, chitosan, alginate-based material, polyhydroxyalkanoate and polydioxanone.

The degradable coating 14 is also mixed with one of hydrophilic substances, antibacterial and anti-inflammatory substances and anticoagulant substances, wherein the hydrophilic substance is one of polyvinylpyrrolidone, sodium hyaluronate and hydrogel; the antibacterial and anti-inflammatory substance is one of triclosan, silver sulfadiazine salt, sirolimus and rifampicin; and the anticoagulant substance is heparin sodium, so as to reduce the stimulation of the ureteral stent 1 to human bodies and further avoid impurities such as calculus scale or bacterial biofilm on the surface of the ureteral stent 1.

The ureteral stent 1 includes a stent body 11 and a forward J-shaped tube 12. The stent body 11 is tubular, and one end far from the discharged body 3 is connected with the forward J-shaped tube 12. A drainage channel 16 runs through the stent body 11 and the forward J-shaped tube 12, and the drainage channel 16 is located in the nondegradable base layer 13 and runs through the front and back. There are also several drainage holes 17 on the stent body 11 and the forward J-shaped tube 12 to communicate with the outside and the drainage channel 16. Among them, the drainage channel 16 and the drainage hole 17 can fully drain urine in the renal pelvis and ureter 6, so as to play a role in drainage and dredging.

There are several marking scales for length measurement outside the stent body 11. The hardness of the forward J-shaped tube 12 and the stent body 11 gradually becomes softer, and the diameters of the forward J-shaped tube 12 and the stent body 11 gradually decrease. Wherein, the forward J-shaped tube 12 is located at the renal pelvis side of human body, while the other end of the stent body 11 is located at the bladder side of human body, that is, the hardness of the ureteral stent 1 gradually softens from the renal pelvis end to the bladder end, so that the ureteral stent 1 can be pushed through the guide wire without wrinkling on the premise of ensuring the stable fixation of the forward J-shaped tube 12 located in the renal pelvis, and at the same time, the stent body 11 has the maximum softness, and further increases the natural space of the ureter after the ureteral stent is placed, which is beneficial to the natural drainage of the ureter. Specifically, the forward J-shaped tube 12 can be made of polyurethane with a hardness of 95, and the stent body 11 can be made of softer polyurethane or silica gel. The diameter of the J-shaped head at the end of renal pelvis is F8-F10, and gradually tapers down to F4-F5. For the marking scale, a first positioning ring is arranged at the joint of the stent body 11 and the forward J-shaped tube 12, a second positioning ring is arranged at a distance of 15 cm from the first positioning ring, and then the next positioning ring is arranged every 5 cm, and a ruler scale can be arranged every 1 cm between two adjacent positioning rings. At least one of the ring number, ring width or ring color of each positioning ring is different. With this design, when the ureteral stent 1 is implanted, it can avoid too many marking rings, which are too complicated and difficult to see. In addition, it can cooperate with the guide wire that can measure the length of ureter during operation, which is convenient for individualized selection of ureteral stent 1 with appropriate length. At the same time, the implantation depth of the ureteral stent 1 can be accurately known under direct vision during operation, and the end of the straight opening of the ureteral stent 1 is ensured to be above the bladder opening of the ureter 6.

The degradable fixture 4 is a degradable wire, which is a binary copolymer formed by the polymerization of glycolide and lactide, and the time-limited fixation of the discharged body 3 and the time-limited folding of the traction wire 2 are completed by the degradable wire alone. Specifically, when the degradable wire is used, it is in the form of a knot, on the one hand, the discharged body 3 is bound and fixed on the end of the traction wire 2 close to the ureteral stent 1, and on the other hand, the traction wire is bound into a folded shape. Wherein, the binary copolymer formed by the polymerization of glycolide and lactide may be Vicryl Rapide, Polyglactin 910 produced by Johnson & Johnson Company, which is a copolymer of 90% glycolide and 10% L-lactide, with a suture tension maintained for 5 days, an absorption time in vivo being 42 days, which can make discharge time of the discharged body 3 in the bladder be 30-45 days approximately. Moreover, because this suture is a multi-strand braided wire, it is 10-30 strands after splitting, which is 10-50 zeros, so one of them can be used to bind the traction wire 2 and the discharged body 3, so that the discharged body 3 can be discharged in the bladder for about 15-30 days. This method can meet the needs of most clinical surgical patients. In addition, the degradable fixing wire 4 has 10-50 zeros and a length of 1-4 cm, which is thinner than the hair, and produces few degradation products in the bladder, which is easy to be discharged with urine and has no great discomfort to human body. It should also be emphasized that the user can further control the degradation time of the degradable wire in urine by adjusting the thickness of the degradable wire, the distribution of different components in the degradable material (such as increasing the content of glycolide), the molecular weight and other factors. At the same time, it is believed that with the further research of degradable materials, the degradation time of the degradable wire can be more accurate, and then the time of taking out the ureteral stent 1 without surgery can be more accurate. In addition, the traction wire 2 is bound in a 6-strand shape, and the folded traction wire 2 is 3-4 cm, which can effectively improve the convenience of surgical placement and effectively avoid discomfort caused by the traction wire 2 being discharged into the posterior urethra in advance in the bladder.

The length of the traction wire 2 is longer than the length of the human urethra (20-30 cm for men and 8-15 cm for women), so as to meet the requirement of discharging the discharged body 3 smoothly. The traction wire 2 is non-absorbent and nondegradable, and some points on the line segment of the traction wire 2 are simultaneously fixed by degradable wires and/or medical glue to be folded. The traction wire 2 can be made of unattractive materials such as polypropylene, and the surface of the traction wire 2 can also be provided with an antibacterial coating.

The surface of the discharged body 3 is smooth and provided with two communicating through holes 31, the density of the discharged body 3 is higher than that of water, both the traction line 2 and the degradable wire can pass through a smaller through hole to bind the discharged body 3, and the aperture size of the other through hole 31 can also be used for the loach guide wire (a model of 038/150) or zebra guide wire to pass through. In this way, the discharged body 3 and the ureteral stent 1 can pass through the guide wire together during surgical placement, and then be implanted into the body through the push rod, thus effectively improving the convenience of operation. Wherein, the discharged body 3 can take various shapes, preferably a spherical shape without obvious foreign body sensation in the body. Specifically, the discharged body 3 is a small round ball with holes made of 304 stainless steel, which has little influence on human body, and its outer diameter is 2-5 mm.

The Work Process of the Present Application:

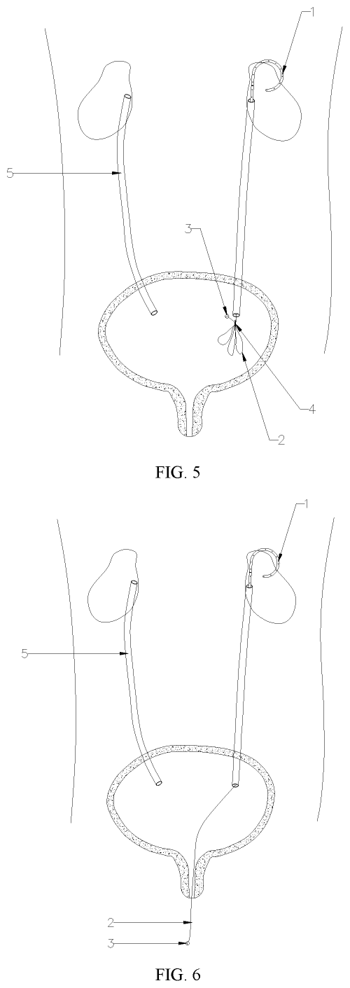

Referring to FIG. 5, in the installation process, a guide wire capable of measuring the length of the ureter was first placed in the ureter 6, and the length of the ureter 6 was measured under the direct vision of a ureteroscope, and then a ureteral stent with an appropriate length and an in-bladder retaining structure 18 was selected according to the specific conditions of hydronephrosis, ureter 6 and bladder during the operation. Taking the single J-shaped ureteral stent 1 without the in-bladder retaining structure 18 as an example, the traction wire 2 was passed through a small through hole of the discharged body 3 and the discharged body 3 was bound, and then some points on the line segment of the traction wire 2 were bound with a degradable fixture 4 (Vicryl Rapide, Polyglactin 910 with a thickness of about 10 zeros) and the discharged body 3 was fixed at the same time, so that the traction wire 2 was folded and the discharged body 3 was arranged close to the ureteral stent 1. Subsequently, the ureteral stent 1 and the other large through hole of the discharged body 3 simultaneously pass through the outside of the guide wire, and then slowly advanced along the guide wire, so that the forward J-shaped tube 12 was placed at the renal pelvis of the patient, and the end of the stent body 11 was placed in the opening of bladder and ureter of the patient, and at the same time, the folded traction wire 2, the discharged body 3 and the degradable fixture 4 were placed inside the bladder at one time. Only the traction line 2 passed through the opening of bladder and ureter, therefore the anti-reflux effect of muscles at the opening of bladder and ureter was not affected. Wherein, the ruler scale between the positioning ring on the ureteral stent 1 and the adjacent positioning ring could help doctors accurately judge the implantation depth of the ureteral stent 1 under direct vision. During use, the nondegradable degradable fixture 4 could prevent the discharged body 3 from being discharged from the body prematurely under the action of gravity and the impact force of urine during urination.

Referring to FIG. 6, when the degradable fixture 4 was degraded, the folded traction wire 2 and the discharged body 3 were released, and the discharged body 3 would be discharged smoothly under the action of gravity and the impact force of urine. At this time, medical staff or patients could pull the discharged body 3, so as to pull the ureteral stent 1 connected with the traction wire 2 out of the body together, thus avoiding secondary cystoscopy surgery.

### Embodiment 2

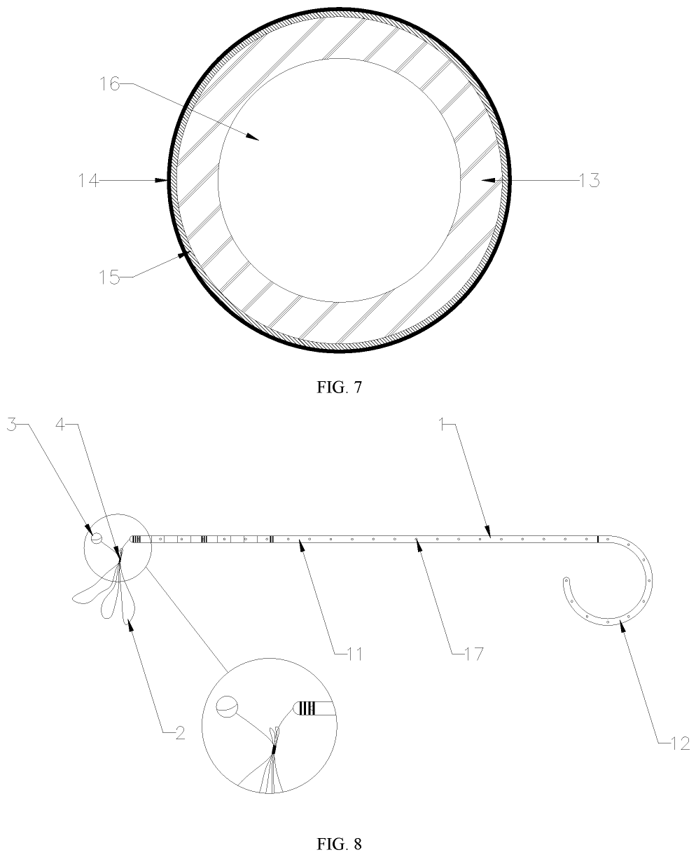

Referring to FIG. 7, a hydrophilic coating 15 was further provided between the nondegradable base layer 13 and the degradable coating 14, that is, the ureteral stent 1 had a three-layer structure.

The hydrophilic coating 15 was composed of one of polyacrylamide, polyvinylpyrrolidone, polyoxyethylene, hydrogel and sodium hyaluronate. The added hydrophilic coating 15 could make the surface of the ureteral stent 1 still have good hydrophilicity after the degradable coating 14 was degraded, thereby improving the implantation comfort of the ureteral stent 1.

Others were the same as in Embodiment 1.

### Embodiment 3

Referring to FIG. 8, the degradable fixture 4 was medical glue, and the medical glue was a-cyanoacrylate. The time-limited fixation of the discharged body 3 and the time-limited folding of the traction wire 2 were completed by medical glue alone. Specifically, on the one hand, the medical glue sticked the discharged body 3 to the end of the traction wire 2 close to the ureteral stent 1, and on the other hand, sticked the traction wire 2 into a folded shape.

Others were the same as in Embodiment 1.

### Embodiment 4

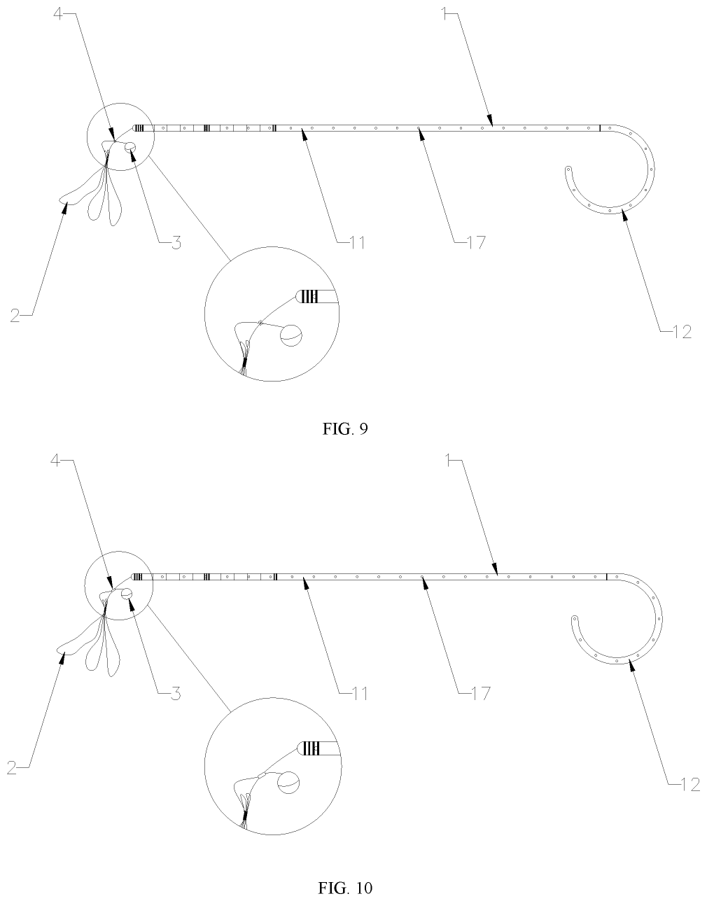

Referring to FIG. 9, the degradable fixture 4 was a degradable wire and medical glue, wherein the degradable wire was a binary copolymer formed by the polymerization of glycolide and lactide, and the medical glue is a-cyanoacrylate. The time-limited fixation of the discharged body 3 and the time-limited folding of the traction wire 2 were jointly completed by the degradable wire and the medical glue. Specifically, the degradable wire bound and fixes the discharged body 3 on one end of the traction wire 2 close to the ureteral stent 1, and the medical glue simultaneously sticked the traction wire 2 into a folded shape.

Others were the same as in Embodiment 1.

### Embodiment 5

Referring to FIG. 10, the degradable fixture 4 was a degradable porous module and medical glue, wherein the degradable porous module was a binary copolymer formed by polymerization of glycolide and lactide, a terpolymer formed by polymerization of glycolide, lactide and caprolactone or an alginate polymer, and the medical glue was a-cyanoacrylate. The time-limited fixation of the discharged body 3 and the time-limited folding of the traction wire 2 were jointly completed by the degradable perforated module and the medical glue. Specifically, the traction wire 2 could pass through the tube hole of the degradable perforated module to bind and fix the discharged body 3 on one end of the traction wire 2 close to the ureteral stent 1, and the medical glue simultaneously sticked the traction wire 2 into a folded shape. Wherein, alginate degraded quickly after about 72 hours, which was suitable for patients with short-term indwelling ureteral stents. While the binary copolymer formed by the polymerization of glycolide and lactide and the ternary copolymer formed by the polymerization of glycolide and lactide and caprolactone could control the specific degradation time of the degradable porous module by controlling the content of glycolide and its molecular weight.

Others were the same as in Embodiment 1.

### Embodiment 6

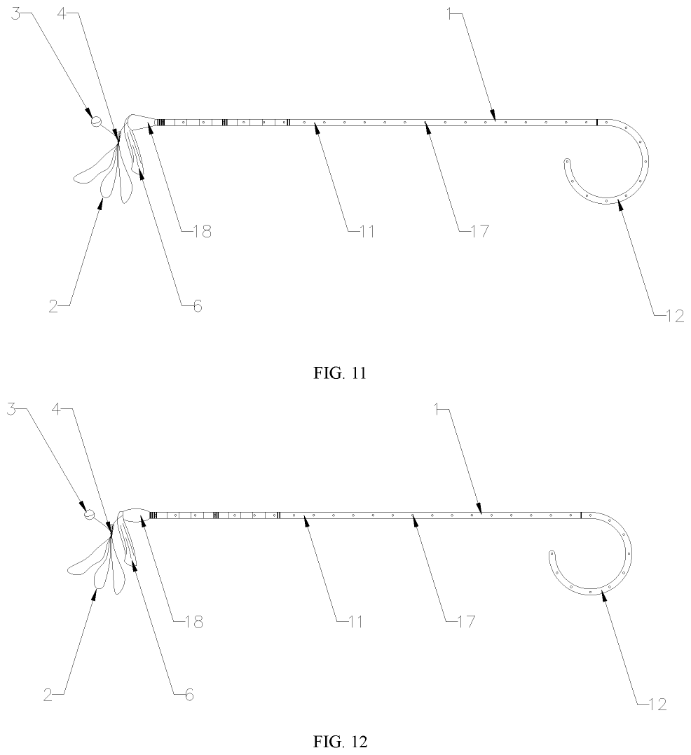

Referring to FIG. 11, one end of the stent body 11 near the discharged body 3 was further provided with a bladder retaining structure 18, which was a flared tube. Compared with the existing curled retaining structure (pigtail shape), this bladder retaining structure can effectively reduce the stimulation to the triangular area of the bladder, and at the same time reduce the phenomenon that the ureteral stent 11 moves up due to the reflux peristalsis of the ureter 6 or bladder contraction.

The other end of the bladder retaining structure 18 was also connected with one end of the anti-reflux water-retaining soft sleeve 6, and the length of the anti-reflux water-retaining soft sleeve 6 was not less than twice the width of the bladder retaining structure 18.

There were several marking scales for length measurement outside the stent body 11. The hardness of the forward J-shaped tube 12, the stent body 11 and the bladder retaining structure 18 gradually became soft, and the diameters of the forward J-shaped tube 12 and the stent body 11 gradually decreased. The diameter of the J-shaped head at the end of renal pelvis was F8-F10, and gradually tapered down to F4-F5. With this design, the stent body 11 can be guaranteed to have the maximum flexibility, and at the same time, the natural space of the ureter after the ureteral stent was indwelled is further increased, which was beneficial to the natural drainage of the ureter and reduced discomfort.

Others were the same as in Embodiment 1.

Under normal circumstances, the bladder, as a urine storage organ, has a receptive relaxation function, and its internal pressure is stable at 0-15 cm water column. In a certain volume range, the bladder has no detrusor contraction and the internal pressure is stable, while the normal renal pelvis pressure is less than 15 cm water column. In addition, the ureter 6 is a muscular tubular structure, and its function is to transport urine. After the traditional ureteral stent is placed, because the bladder does not contract during the urine storage period, the urine produced by the kidney can be smoothly discharged into the bladder from the renal pelvis, the ureteral stent and the ureter 6, so that the patient has no discomfort of waist swelling except the stimulation of the ureteral stent itself. However, when urinating, the bladder pressure will increase, generally reaching 40 cm water column or even 70-100 cm water column. At this time, due to the pressure increase, urine easily flows back to the renal pelvis through the ureteral stent and ureter 6, and the corresponding pressure in the bladder will also be transmitted to the renal pelvis, which will lead to pain and discomfort in the affected side of the patient.

For this embodiment, during the urine storage period, the anti-reflux water-retaining soft sleeve 6 was in a free state, while during urination, the bladder pressure rose, and the anti-reflux water-retaining soft sleeve 6 could cover the orifice of the flared tube under the action of the bladder pressure, so that the inner space of the ureteral stent 1 was completely separated from the inner space of the bladder, thereby preventing the urine from flowing back.

### Embodiment 7

Referring to FIG. 12, one end of the stent body 11 near the discharged body 3 was further provided with a bladder retaining structure 18, which was an elliptical tube.

The other end of the bladder retaining structure 18 was also connected with one end of the anti-reflux water-retaining soft sleeve 6, and the length of the anti-reflux water-retaining soft sleeve 6 was not less than twice the width of the bladder retaining structure 18.

There were several marking scales for length measurement outside the stent body 11. The hardness of the forward J-shaped tube 12, the stent body 11 and the bladder retaining structure 18 gradually became soft, and the diameters of the forward J-shaped tube 12 and the stent body 11 gradually decreased.

Others were the same as in Embodiment 1.

During the urine storage period, the anti-reflux water-retaining soft sleeve 6 was in a free state, while during urination, the bladder pressure rose, and the anti-reflux water-retaining soft sleeve 6 could cover the orifice of the elliptical tube under the action of the bladder pressure, so that the space in the tube of the ureteral stent 1 was completely separated from the space in the bladder, thereby preventing the urine from flowing back.

### Embodiment 8

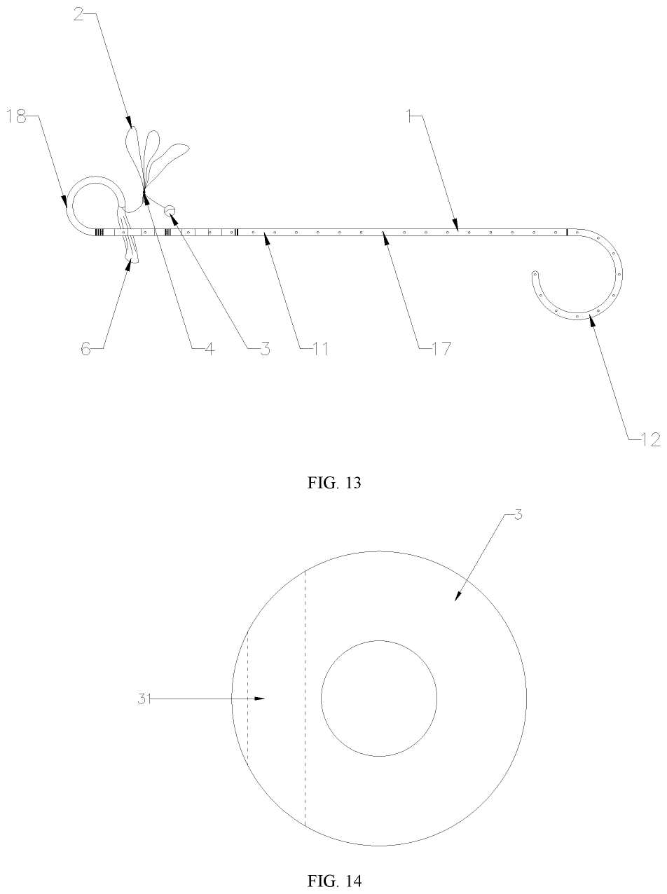

Referring to FIG. 13, one end of the stent body 11 near the discharged body 3 was further provided with a bladder retaining structure 18, which was an inverted J-shaped tube, and the size of the ring of the inverted J-shaped tube was smaller than that of the forward J-shaped tube 12.

The other end of the bladder retaining structure 18 was also connected with one end of the anti-reflux water-retaining soft sleeve 6, and the length of the anti-reflux water-retaining soft sleeve 6 was not less than twice the width of the bladder retaining structure 18.

There were several marking scales for length measurement outside the stent body 11. The hardness of the forward J-shaped tube 12, the stent body 11 and the bladder retaining structure 18 gradually became soft, and the diameters of the forward J-shaped tube 12 and the stent body 11 gradually decreased.

Others were the same as in Embodiment 1.

During the urine storage period, the anti-reflux water-retaining soft sleeve 6 was in a free state, while during urination, the bladder pressure rises, and the anti-reflux water-retaining soft sleeve 6 could cover the orifice of the forward J-shaped tube 12 under the action of the bladder pressure, so that the inner space of the ureteral stent 1 was completely separated from the inner space of the bladder, thereby preventing the urine from flowing back.

### Embodiment 9

Referring to FIG. 14, the surface of the discharged body 3 was smooth and provided with two communicating through holes 31. One of the through holes was used to tie the discharged body with a traction wire, and the other large through hole could be implanted into the bladder by sleeving the discharged body into the guide wire push rod through the loach guide wire or zebra guide wire.

Others were the same as in Embodiment 1.

The above embodiment is an explanation of the present application, not a limitation of the present application, and any solution after simple transformation of the present application belongs to the protection scope of the present application.

What is claimed is:

1. A controllable operation-free take-out ureteral stent with a degradable coating, comprising: a ureteral stent, a traction wire, a discharged body, and a degradable fixture, wherein the discharged body is fixed on a first end of the traction wire, and the degradable fixture fixes the discharged body on a second end of the traction wire and is arranged adjacent to an end of the ureteral stent within a limited period of time; the ureteral stent comprises a nondegradable base layer and the degradable coating, wherein the nondegradable base layer and the degradable coating are sequentially arranged from an inside to an outside.

2. The controllable operation-free take-out ureteral stent with the degradable coating according to claim 1, wherein the nondegradable base layer is made of polyurethane or silica gel, and the degradable coating layer is made of a degradable material selected from polylactic acid, polyglycolic acid, polylactic-glycolic acid copolymer, chitosan, alginate-based material, polyhydroxyalkanoate, and polydioxanone.

3. The controllable operation-free take-out ureteral stent with the degradable coating according to claim 2, wherein a hydrophilic coating is arranged between the nondegradable base layer and the degradable coating, and the hydrophilic coating is made of one of polyacrylamide, polyvinylpyrrolidone, polyoxyethylene, hydrogel, and sodium hyaluronate.

4. The controllable operation-free take-out ureteral stent with the degradable coating according to claim 1, wherein the ureteral stent comprises a stent body and a forward J-shaped tube, wherein the stent body is tubular and a first end of the stent body far from the discharged body is connected to the forward J-shaped tube; a drainage channel runs through between the stent body and the forward J-shaped tube, the drainage channel is located in the nondegradable base layer and runs through a front and a back, and a plurality of drainage holes communicating with an outside and the drainage channel are respectively arranged on the stent body and the forward J-shaped tube.

5. The controllable operation-free take-out ureteral stent with the degradable coating according to claim 4, wherein a second end of the stent body adjacent to the discharged body is further provided with a bladder retaining structure, wherein the bladder retaining structure is a flared tube, an elliptical tube, or an inverted J-shaped tube, and a ring size of the inverted J-shaped tube is smaller than a ring size of the forward J-shaped tube.

6. The controllable operation-free take-out ureteral stent with the degradable coating according to claim 5, wherein the other end of the bladder retaining structure is connected to an end of an anti-reflux water-retaining soft sleeve, and a length of the anti-reflux water-retaining soft sleeve is greater than or equal to twice a width of the bladder retaining structure.

7. The controllable operation-free take-out ureteral stent with the degradable coating according to claim 5, wherein a plurality of marking scales for a length measurement are arranged outside the stent body, and a hardness of the forward J-shaped tube, the stent body and the bladder retaining structure gradually becomes softer, and diameters of the forward J-shaped tube and the stent body gradually become smaller.

8. The controllable operation-free take-out ureteral stent with the degradable coating according to claim 1, wherein the degradable fixture is one or more of a degradable wire, a degradable perforated module, and a medical glue; the degradable wire and the degradable perforated module are binary copolymers formed by a polymerization of glycolide and lactide, terpolymers or alginate polymers formed by a polymerization of glycolide, lactide, and caprolactone, and the medical glue is a-cyanoacrylate.

9. The controllable operation-free take-out ureteral stent with the degradable coating according to claim 8, wherein a length of the traction wire is longer than a length of a human urethra, the traction wire is a non-absorbent and nondegradable wire, and some points on a line segment of the traction wire are simultaneously fixed by the degradable wire and/or the medical glue to be folded.

10. The controllable operation-free take-out ureteral stent with the degradable coating according to claim 9, wherein a surface of the discharged body is smooth and provided with a plurality of through holes, a density of the discharged body is higher than a density of water; the traction wire and the degradable wire are configured to pass through the plurality of through holes to bind the discharged body, and a largest through hole of the plurality of through holes has a hole size allowing a loach guide wire or a zebra guide wire to pass through.
